(19) United States
(12) Patent Application Publication
(10) Pub. No.: US 2002/0182635 A1
(43) Pub. Date: Dec. 5, 2002

(54) Methods of screening for LTRPC2 modulators

(76) Inventors: Reinhold Penner, Honolulu, HI (US); Andrea Fleig, Honolulu, HI (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 10/007,706

(22) Filed: Nov. 13, 2001

Related U.S. Application Data
(60) Provisional application No. 60/248,442, filed on Nov. 13, 2000.
(60) Provisional application No. 60/254,528, filed on Dec. 8, 2000.

(51) Int. Cl. G01N 33/53; G01N 33/567; G01N 33/543

(52) U.S. Cl. 435/7.1; 436/518; 435/7.2

(57) ABSTRACT

The present invention relates to the identification and isolation of a novel family of ADP ribose (“ADPR) regulated calcium transmembrane channel polypeptides designated herein as “LTRPC2” (Long Transient Receptor Potential Channel). Channels comprising these polypeptides open in response to concentrations of cytoplasmic ADPR in the micromolar range, exhibit enhanced activity in the presence of high intracellular levels of calcium, and do not respond to depletion or reduction in intracellular calcium stores. The invention further relates to the methods of utilizing LTRPC2 for binding, and the methods for modulating LTRPC2 activity and for measuring LTRPC2 permeability. The invention further relates to the methods of modulating expression of LTRPC2. PATENT

### FIELD OF THE INVENTION

The present invention relates to the identification and isolation of a novel family of ADP ribose (“ADPR) regulated calcium transmembrane channel polypeptides designated herein as “LTRPC2” (Long Transient Receptor Potential Channel). Channels comprising these polypeptides open in response to concentrations of cytoplasmic ADPR in the micromolar range, exhibit enhanced activity in the presence of high intracellular levels of calcium, and do not respond to depletion or reduction in intracellular calcium stores. The invention further relates to the recombinant nucleic acids that encode LTRPC2 and the methods of utilizing LTRPC2 to bind candidate bioactive agents for modulating LTRPC2 activity and for measuring LTRPC2 permeability to multivalent cations. The invention further relates to methods of modulating the cellular expression of the recombinant nucleic acids that encode LTRPC2.

### BACKGROUND OF THE INVENTION

Ion channels are transmembrane multi-subunit proteins embedded in the cellular plasma membranes of living cells which permit the passage of specific ions from the extracelluar side of the plasma membrane to the intracellular region of the cell. Specific ion transport is facilitated by a central aqueous pore which is capable of opening and closing due to changes in pore conformation. When the ion gate is open, ions flow freely through the channel. When the ion gate is closed, ions are prevented from permeating the channel. Ion channels are found in a multitude of multicellular eukaryotic species and in a myriad of different cell types. Ion channels may be either voltage-gated or ligand-gated. Channel gating is the process by which a particular channel is either open or closed. An ion channel may be capable of occupying a range of different “open” or “closed” states. The gating process may therefore require a particular sequence of transition states or inclusion of alternative transition states before a channel attains a particular level of gating. The gating process is modulated by a substance or agent, which in some way alters or affects the manner in which the channel opens or closes. A channel may be gated by a ligand such as a neurotransmitter, an internal primary or secondary messenger, or other bioactive agent. The ligand either attaches to one or more binding sites on the channel protein or attaches to a receptor that is associated with the channel. If the channel is voltage-gated, changes in the membrane potential trigger channel gating by conformational changes of charged elements within the channel protein. Whether a channel is ligand-gated or voltage-gated, a change in one part of the channel produces an effect in a different part of the channel which results in the opening or closing of a permeant pathway.

### SUMMARY OF THE INVENTION

The invention relates to the identification, isolation and use of a novel family of ADPR regulated calcium transmembrane channel polypeptides designated herein as “LTRPC2” (Long Transient Receptor Potential Channel) which open in response to increasing concentrations of cytoplasmic ADPR in the micromolar range, exhibit enhanced activity in the presence of high intracellular levels of calcium, and do not respond to depletion or reduction in intracellular calcium stores. The invention further relates to the recombinant nucleic acids that encode LTRPC2 and the methods of utilizing LTRPC2 to bind candidate bioactive agents for modulating LTRPC2 activity and for measuring LTRPC2 permeability to multivalent cations. The invention further relates to methods of modulating the cellular expression of the recombinant nucleic acids that encode LTRPC2.

One embodiment of the invention provides methods for screening for candidate bioactive agents that bind to LTRPC2. In this method, LTRPC2, or a fragment thereof, is contacted with a candidate agent, and it is determined whether the candidate agent binds to LTRPC2. An embodiment of the invention provides for contacting LTRPC2 with a library of two or more candidate agents and then determining the binding of one or more of the candidate agents to LTRPC2.

In a further embodiment, LTRPC2 comprises an ion channel and the candidate agent(s) that bind the LTRPC2 channel modulate the multivalent cationic permeability of the LTRPC2 channel. In some embodiments, the candidate agent(s) that bind LTRPC2, open the LTRPC2 channel. In still another embodiment, the candidate agents that bind LTRPC2, close the LTRPC2 channel.

In some embodiments the LTRPC2 channel is in a recombinant cell which comprises a recombinant nucleic acid encoding LTRPC2, an inducible promoter which is operably linked to the recombinant nucleic acid, and a multivalent cation indicator, such as fura-2. The recombinant cell is induced to express LTRPC2 and it is then contacted with a solution comprising a multivalent cation together with a candidate agent. In another embodiment, the recombinant cell is contacted with a candidate agent prior to being contacted with a multivalent cation. Intracellular levels of the multivalent cation are detected using the multivalent cation indicator. In some embodiments, the candidate agent increases the multivalent cation permeability of the LTRPC2 channel. In other embodiments, the candidate agent decreases the multivalent cation permeability of the LTRPC2 channel. In a preferred embodiment, the multivalent cation indicator comprises a fluorescent molecule. In a more preferable embodiment of the invention, the multivalent cation indicator comprises fura-2. In an alternate embodiment, the production of LTRPC2 channel is induced and the multivalent cation intracellular levels are detected in the presence of a candidate agent. That level is compared to the multivalent cation intracellular level detected in an uninduced recombinant cell either in the presence or absence of a candidate agent.

It is another object of the invention to provide methods for measuring the multivalent ion permeability of an LTRPC2 channel. In this method, a recombinant cell is provided, which comprises a recombinant nucleic acid encoding LTRPC2, a promoter, either constitutive or inducible, preferably inducible, which is operably linked to the recombinant nucleic acid, and an intracellular cation indicator. The recombinant cell is contacted with a solution comprising a multivalent cation that selectively interacts with the indicator to generate a signal. Intracellular levels of the multivalent cation are then measured when LTRPC2 is expressed by detecting the indicator signal. This measurement is compared to endogenous levels in which recombinant LTRPC2 is not expressed. In a broader embodiment, the cell is not limited to a recombinant LTRPC2 expressing cell, but can comprise any cell capable of being used with any recombinantly expressed channel protein for determining agents which modulate the activity of the channel. In a preferred embodiment the multivalent cation indicator comprises a fluorescent molecule such as fura-2. In some embodiments the modulating activity of a candidate bioactive agent which contacts the recombinant cell together with the multivalent cation agent increases the multivalent cation permeability of the LTRPC2 channel, in others it decreases it. In further embodiments the modulating activity of a candidate bioactive agent which contacts the recombinant cell prior to contact with the multivalent cation agent increases the multivalent cation permeability of the LTRPC2 channel, in others it decreases it.

It is further an object of the invention to provide methods for screening for candidate bioactive agents that are capable of modulating expression of LTRPC2. In this method, a recombinant cell is provided which is capable of expressing a recombinant nucleic acid encoding LTRPC2, a fragment thereof, including in some embodiments the 5′ and/or 3′ expression regulation sequences normally associated with the LTRPC2 gene. The recombinant cell is contacted with a candidate agent, and the effect of the candidate agent on LTRPC2 expression is determined. In some embodiments, the candidate agent may comprise a small molecule, protein, polypeptide, or nucleic acid (e.g., antisense nucleic acid). In another embodiment of the invention, LTRPC2 expression levels are determined in the presence of a candidate bioactive agent and these levels are compared to endogenous LTRPC2 expression levels.

Another aspect of the invention is a recombinant LTRPC2 protein or fragment thereof having the sequence of amino acids from 1 through about 1503 of SEQ ID NO:1 (FIG. 6) where LTRPC2 is a transmembrane channel polypeptide which opens in response to concentrations of intracellular ADPR in the micromolar range, exhibits enhanced activity in the presence of high intracellular levels of calcium, and does not respond to depletion or reduction in intracellular calcium stores.

Another aspect of the invention is an isolated recombinant nucleic acid molecule having at least 80% sequence identity to a DNA molecule encoding a recombinant LTRPC2 protein or fragment thereof having the sequence of amino acids from 1 through about 1503 of SEQ ID NO:1 (FIG. 6) and having GenBank Accession No. BAA34700. An embodiment of the invention is a recombinant nucleic acid molecule comprising sequences from 446 through about 4957 of SEQ. ID NO.3 (FIG. 8) and having GenBank Accession No. AB001535.

Another aspect of the invention is an isolated recombinant nucleic acid molecule comprising an LTRPC2 gene comprising the sequence from 1 through about 6220 of SEQ ID NO: 3 (FIG. 8) and having GenBank Accession No. AB001535, wherein said recombinant nucleic acid molecule encodes a recombinant LTRPC2 protein or any preferred fragments thereof having the sequence of amino acids from 1 through about 1503 of FIG. 6 (SEQ ID NO: 1) or a sequence which is at least 80% identical to said protein sequence.

In a further embodiment of the invention, LTRPC2 comprises polypeptides having an amino acid sequence comprising from 1 through about 1503 amino acids having SEQ ID NO:1 (FIG. 6). In a further embodiment, LTRPC2 is encoded by nucleic acid sequences of nucleotides comprising nucleotides from about 446 through about 4957 of SEQ ID NO:3 (FIG. 8).

### BRIEF DESCRIPTION OF THE DRAWINGS

FIG. 1 depicts the protein sequence analysis of LTRPC2. FIG. 1(A) is a schematic of LTRPC2 structural motifs based on alignments of various related proteins including MLSN-1, LTRPC7, MTR-1, and the C. elegans proteins C05C12.3, T01H8.5, and F54D1.5. Bottom: ClustalW alignment oftheNUDT9 homology region of LTRPC2, EEED8.8, and NUDT9. The putative signal peptide or anchor found in NUDT9 is double underlined (prediction based on SignalP2.0 analysis of the NUDT9 amino acid sequence). The Nudix box region is boxed by thick lines. FIG. 1(B) shows a qualitative RT-PCR analysis of LTRPC2 and NUDT9 expression in a selection of human tissues. Primers specific for either LTRPC2 (138 bp band) or NUDT9 (252 bp band) were used to prime PCR reactions from cDNA libraries prepared from the indicated tissues. A lack of band of the correct size was interpreted as negative (−), and the presence of a band was interpreted as positive (+). A 4.0 kb partial LTRPC2 cDNA (including the 5′ end, and terminating at the internal NotI site) was subsequently cloned from the same leukocyte cDNA library used for these PCR reactions. Multiple NUDT9 cDNAs were obtained from a single screening of the same spleen cDNA library used for these PCR reactions.

FIG. 2 demonstrates the bacterial expression and enzymatic characterization of NUDT9 and LTRPC2 NUDT9-H. FIG. 2(A) is an SDS-PAGE analysis of NUDT9 and NUDT9-H. Crude bacterial fractions before induction (non), after induction (I), and purified preparations (P) of NUDT9 and NUDT9-H were analyzed by SDS-PAGE and coomassie blue staining. FIG. 2(B) is a characterization of the enzymatic activity of NUDT9 and NUDT9-H. Purified preparations of NUDT9 and NUDT9-H were screened for Nudix-type activity towards a panel of substrates as described in the methods section. Kₘ and Vₘₐₓ were calculated by non-linear regression analysis of Lineweaver-Burke plots. The following compounds, known to be substrates for other members of the Nudix hydrolase family, were not hydrolyzed by NUDT9 and NUDT9-H: deoxy-ADPR, deoxy-CTP, deoxy-GTP, deoxy-TTP, GDP-mannose, ADP-lucose, UDP-glucose, ApₙA (n=2 through 6), NADH, NAD⁺.

FIG. 3 depicts the tetracycline-induced functional expression of LTRPC2 in HEK-293 cells. FIG. 3(A) shows the Wild-type (WT) HEK-293 cells or an HEK-293 cell line with tetracycline-regulated expression of FLAG-LTRPC2 treated for 24 hours with 1 μg/ml of tetracycline were analyzed by northern blot using a human LTRPC2 probe. Recombinant LTRPC2 is revealed as an approximately 5.5 kb mRNA species in tetracycline-treated cells, while no native LTRPC2 transcript is detectable in the untransfected WT 293 cells (even with much longer exposures than that pictured here, no native LTRPC2 transcript was detectable in the WT cells). FIG. 3(B) shows the HEK-293 cell lines with tetracycline-regulated expression of FLAG-LTRPC2 were treated or not for 24 hours with 1 μg/ml of tetracycline. 10⁶ cells were analyzed for expression of a FLAG-reactive protein by anti-FLAG immunoprecipitation/anti-FLAG immunoblotting. Several clones were used in subsequent analyses, and all exhibited indistinguishable biochemical and biophysical behavior. FIG. 3(C) shows the HEK-293 cells with inducible expression of FLAG-LTRPC2 were left untreated or were treated with tetracycline. Pictured is a representative cell observed after tetracycline induction of FLAG-LTRPC2 expression and staining with monoclonal anti-FLAG (red fluorescence), DioC6 (green fluorescence, perinuclear ER) and Hoechst (blue fluorescence, nucleus). Peripheral red staining indicates the presence of LTRPC2 in the plasma membrane. In the absence of tetracycline, there is no detectable FLAG-reactive staining (data not shown). FIG. 3(D) shows a graph which illustrates the temporal development of averaged membrane currents at −80 mV under various experimental conditions. Only tet-induced HEK-293 cells expressing FLAG-LTRPC2 generated large inward currents when perfused with 100 μM ADPR (n=5±sem, filled symbols). The open symbols represent superimposed analyses of responses obtained from (i) wild-type HEK-293 cells (WT) perfused with standard internal solution in the absence of ADPR (n=3±sem); (ii) uninduced cells perfused with standard internal solution in the absence of ADPR (n=5±sem); (iii) uninduced HEK-293 cells perfused with standard solution containing 1 mM ADPR (n=3±sem); (iv) tet-induced HEK-293 cells perfused with standard internal solution without ADPR present (n=4±sem). FIG. 3(E) depicts intracellular perfusion of 300μ ADPR reliably induced almost linear cationic currents with slight outward rectification in LTRPC2-expressing HEK-293 cells. The graph shows, in a representative cell, the concurrent activation of inward and outward currents measured at −80 mV and +80 mV, respectively. The filled symbols indicate the time points at which individual high-resolution data traces were extracted for presentation as I/V curves in FIG. 3(F). FIG. 3(F) shows the current-voltage relationships of ADPR-dependent currents taken from the representative cell in FIG. 3(E) at the indicated times. Ramp currents were recorded in response of a standard voltage ramp stimulus (−100 mV to +100 mV in 50 ms).

FIG. 4 depicts the characterization of ADPR-dependent currents in LTRPC2-expressing in HEK-293 cells. FIG. 4(A) shows the dose-response curve for ADPR-dependent gating of LTRPC2. HEK-293 cells expressing FLAG-LTRPC2 were perfused with defined ADPR concentrations ranging from 10μ to 1 mM, and currents were measured at −80 mV as in FIG. 3(D). The maximum current amplitude of individual cells was derived by analyzing the time course of current development (see e.g., FIGS. 3(C) and 3(D)) and fitting a Boltzmann curve to the rising phase of the developing cationic conductance. Peak current amplitudes were averaged and plotted versus ADPR concentration (n=5 to 12±sem). The averaged data points were fitted with a dose-response curve yielding an apparent EC₅₀ of 90 μM and a Hill coefficient of 9 (fits with constrained Hill coefficients between 4-8 yielded similarly adequate approximations). 91% of all cells perfused with 60 μM ADPR or higher generated currents above control levels (n=38). FIG. 4(B) depicts the kinetics of ADPR-dependent gating of LTRPC2. The temporal development of ADPR-gated currents was assessed as described in FIG. 4(A) by fitting a Boltzmann curve to the rising phase of the developing cationic conductance. The mid-point values of this analysis correspond to the time of half-maximal current activation, and are plotted as a function of ADPR concentration. FIG. 4(C) shows the HEK-293 cells expressing FLAG-LTRPC2 were perfused with 300 μM ADPR. Experiments were performed on cells after 16 h induction, resulting in smaller average current amplitudes At the time indicated by the bar, isotonic NMDG-Cl solution (180 mM NMDG-Cl, 330 mOsm) was applied externally for 20 seconds. The panel shows an average of inward currents from 3 cells±sem. Note that isotonic NMDG solutions are able to completely suppress the current previously carried mainly by Na⁺ ions. FIG. 4(D) shows that LTRPC2 is permeable to calcium. HEK-293 cells expressing FLAG-LTRPC2 were perfused with 100 μM ADPR. 80 seconds into the experiment, and indicated by the bar, isotonic CaCl₂ solution (120 mM CaCl₂, 300 mOsm) was applied externally for 20 seconds. The panel shows an average of inward currents from 3 cells±sem. Note that isotonic Ca²⁺ solutions are able to support about 50% of current previously carried mainly by Na⁺ ions.

FIG. 5 depicts the characterization of endogenous ADPR-dependent current(I_{ADPR}) in human U937 monocytes. FIG. 5(A) shows, in the left lane, Northern blot analysis identifies LTRPC2 as a 6 kb mRNA species in HEK-293 cells treated for 24 hours with 1 μg/ml of tetracycline. In the right lane, the blot identifies LTRPC2 mRNA in native U937 cells. Note that this blot was exposed longer in order to provide optimal detection of the native transcript, hence the marked overexposure of the positive control recombinant transcript in the right lane. FIG. 5(B) depicts the temporal development of inward currents in U937 cells at −80 mV activated by different intracellular concentrations of ADPR in the presence of 10 mM BAPTA (n=4-11 each). FIG. 5(C) depicts the temporal development of inward currents in U937 cells at −80 mV activated by different intracellular concentrations of ADPR while [Ca²⁺]i was buffered to 100 nM (n=5-7 each). FIG. 5(D) depicts the temporal development of inward currents in U937 cells at −80 mV activated by different intracellular concentrations of ADPR in the absence of exogenous buffers (n=5-9 each). FIG. 5(E) shows the dose-response relationships for I_{ADPR} in U937 cells perfused with defined ADPR concentrations while [Ca²⁺]i was buffered to 100 nM (filled symbols) or left to vary freely by omitting exogenous buffers (open symbols). The averaged data points were fitted with a dose-response curve yielding an apparent EC₅₀ of 130 μM and and 40 μM for buffered and unbuffered conditions, respectively (in both cases, Hill coefficients were 8). FIG. 5(F) shows the current-voltage relationship of ADPR currents in U937 cells. Representative current record in response to a voltage ramp ranging from −100 to +100 mV over 50 ms. The record was obtained 100 s after whole-cell establishment from a cell perfused with 100 μM ADPR under unbuffered conditions.

FIG. 6 shows the amino acid sequence of a recombinant LTRPC2 protein comprised of sequences from 1 through about 1503 (SEQ ID NO:1).

FIG. 7 shows the recombinant nucleic acid molecule of an LTRPC2 cDNA encoding sequence (SEQ ID NO:2).

FIG. 8 shows the recombinant nucleic acid molecule of an LTRPC2 gene comprised of nucleic acid sequences from 1 through about 6220 (SEQ ID NO: 3).

### DETAILED DESCRIPTION OF THE PREFERRED EMBODIMENTS

The invention relates, in part, to methods useful in identifying molecules, that bind LTRPC2, which modulate LTRPC2 ion channel-activity, and/or which alter expression of LTRPC2 within cells. The LTRPC2 channels as described herein comprise LTRPC2 polypeptides, which are in turn encoded by LTRPC2 nucleic acids. The ion channels described herein are preferably formed in HEK-293 cells and comprise one or more novel LTRPC2 polypeptides, which exhibit one or more of the unique LTRPC2 properties described herein.

As described herein, the term “LTRPC2” (Long Transient Receptor Potential Channel) refers to a member of the novel family of ADPR regulated calcium transmembrane channel polypeptides. The polypeptides are also defined by their amino acid sequence, the nucleic acids which encode them, and the novel properties of LTRPC2. Such novel properties include opening of the LTRPC2 channel in response to concentrations of intracellular ADPR in the micromolar range, enhancement of activity of the LTRPC2 channel in response to high intracellular levels of calcium, and non-responsiveness of the LTRPC2 channel to a depletion or reduction in intracellular calcium stores. Gating of the LTRPC2 channel begins when intracellular ADPR concentrations are in the 60-100 micromolar range and saturation occurs when ADPR concentrations are in the 300 micromolar range.

The LTRPC2 polypeptides and channels are fundamentally distinct from the “SOC” (Store Operated Channels) and “CRAC” (Calcium Release Activated Channels) polypeptides and channels, disclosed in “Characterization of a Calcium Family,” WO 00/40614, the disclosure of which is expressly incorporated herein by reference. The SOC and CRAC proteins “may be activated upon depletion of Ca²⁺ from intracellular calcium stores” (see WO 00/40614 at page 2) and are further “subject to inhibition by high levels of intracellular calcium” (see WO 00/40614 at page 10). The LTRPC2 channels of the invention, conversely, exhibit enhanced activity in the presence of high intracellular levels of calcium, are not activated by the depletion or reduction in intracellular calcium stores, and open in response to intracellular ADPR concentrations in the micromolar range. SOC and CRAC are not regulated in this manner.

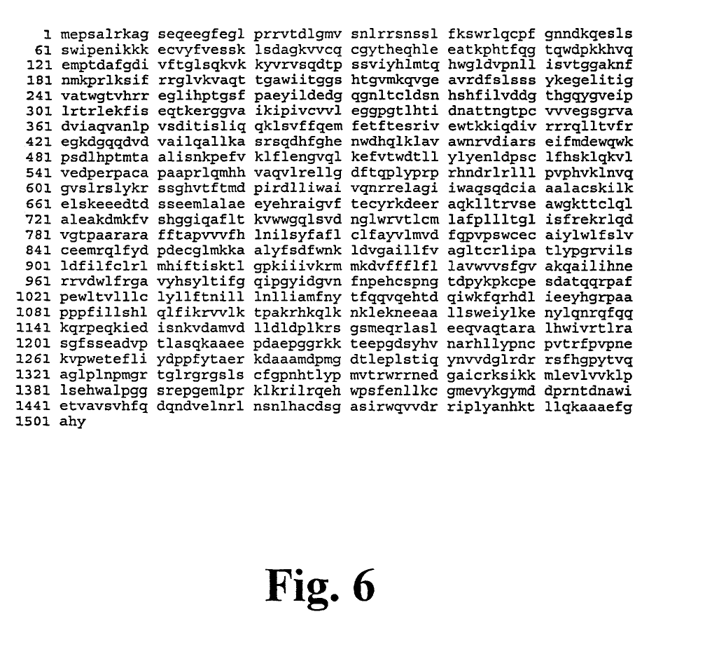

The LTRPC2 polypeptide is a novel member of the LTRPC family. The specific sequence disclosed herein as SEQ ID NO: 1 (FIG. 6) was derived from human spleen cells. However, LTRPC2 is believed to be broadly expressed in tissues from mammalian species, and other multicellular eukaryotes, such as C. elegans.

LTRPC2 can be derived from natural sources or recombinantly modified to make LTRPC2 variants. The term “LTRPC2 sequence” specifically encompasses naturally-occurring truncated or secreted forms (e.g., an extracellular domain sequence), naturally-occurring variant forms (e.g., alternatively spliced forms) and naturally-occurring allelic variants. The native sequence of the LTRPC2 polypeptide from human spleen cells is a full-length or mature native sequence LTRPC2 polypeptide comprising amino acids from 1 through about 1503 of SEQ ID NO:1 (FIG. 6).

The LTRPC2 polypeptide disclosed herein as SEQ ID NO: 1 (FIG. 6) comprises an N-terminal intracellular domain comprising amino acid sequences 1-757; a transmembrane domain comprising sequences 758-1070; a coiled-coil domain comprising sequences 1143-1300; an enzymatic domain with nucleoside diphosphate specificity comprising sequences 1641-1822, and three extracellular domains comprising sequences 774-793, 892-899, and 957-1023.

The LTRPC2 polypeptide of the invention, or a fragment thereof, also includes polypeptides having at least about 80% amino acid sequence identity, more preferably at least about 85% amino acid sequence identity, even more preferably at least about 90% amino acid sequence identity, and most preferably at least about 95% sequence identity with the amino acid sequence of SEQ ID NO:1. Such LTRPC2 polypeptides include, for instance, LTRPC2 polypeptides wherein one or more amino acid residues are substituted and/or deleted, at the N- or C-terminus, as well as within one or more internal domains, of the sequence of SEQ ID NO:1. Those skilled in the art will appreciate that amino acid changes may alter post-translational processes of the LTRPC2 polypeptide variant, such as changing the number or position of glycosylation sites or altering the membrane anchoring characteristics. All LTRPC2 proteins, however, exhibit one or more of the novel properties of the LTRPC2 polypeptides as defined herein.

“Percent (%) amino acid sequence identity” with respect to the LTRPC2 polypeptide sequences identified herein is defined as the percentage of amino acid residues in a candidate sequence that are identical with the amino acid residues of SEQ ID NO:1 (FIG. 6), after aligning the sequences and introducing gaps, if necessary, to achieve the maximum percent sequence identity, and not considering any conservative substitutions as part of the sequence identity. The % identity values used herein are generated by WU-BLAST-2 which was obtained from Altschul et al., Methods in Enzymology, 266:460-480 (1996); http://blast.wustl/edu/blast/README.html. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a further embodiment, the % identity values used herein are generated using a PILEUP algorithm. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of0.10, and weighted end gaps.

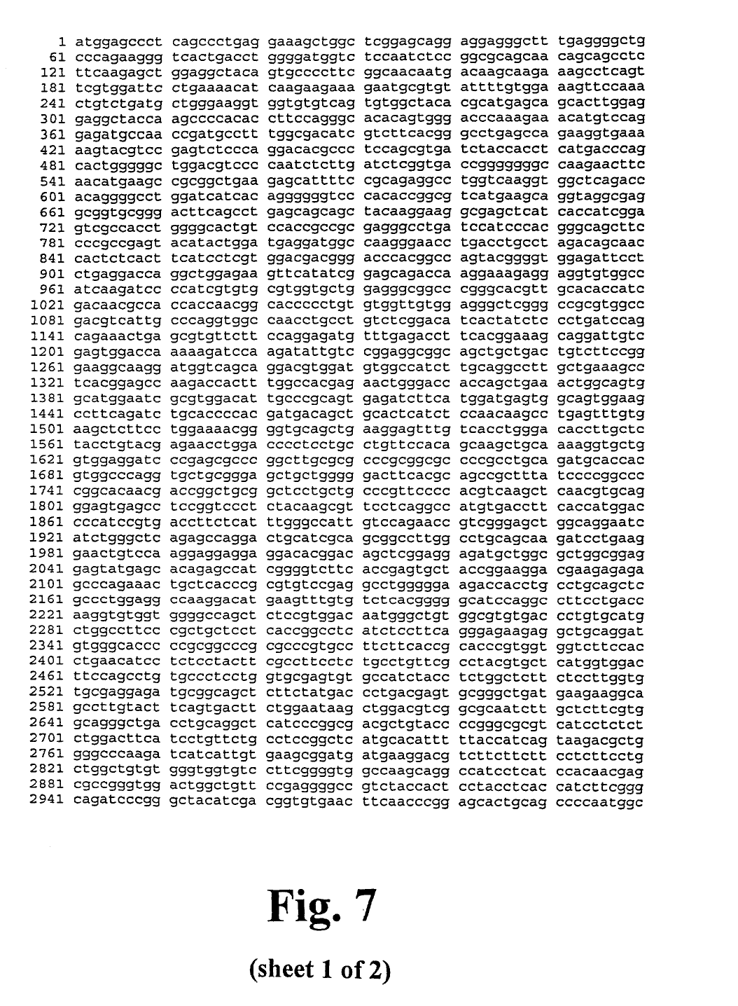

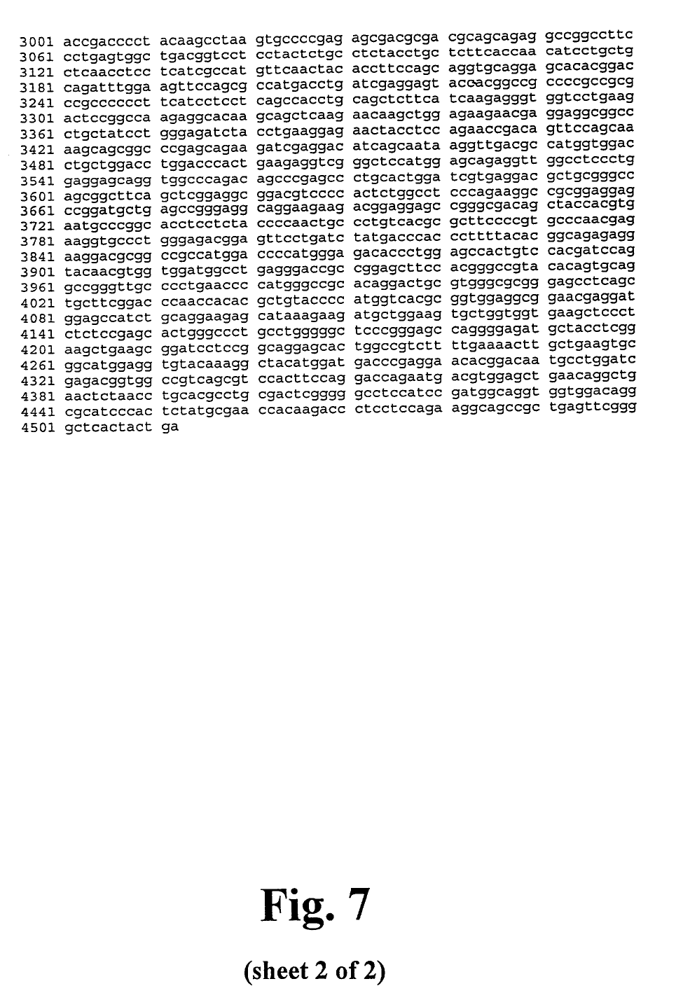

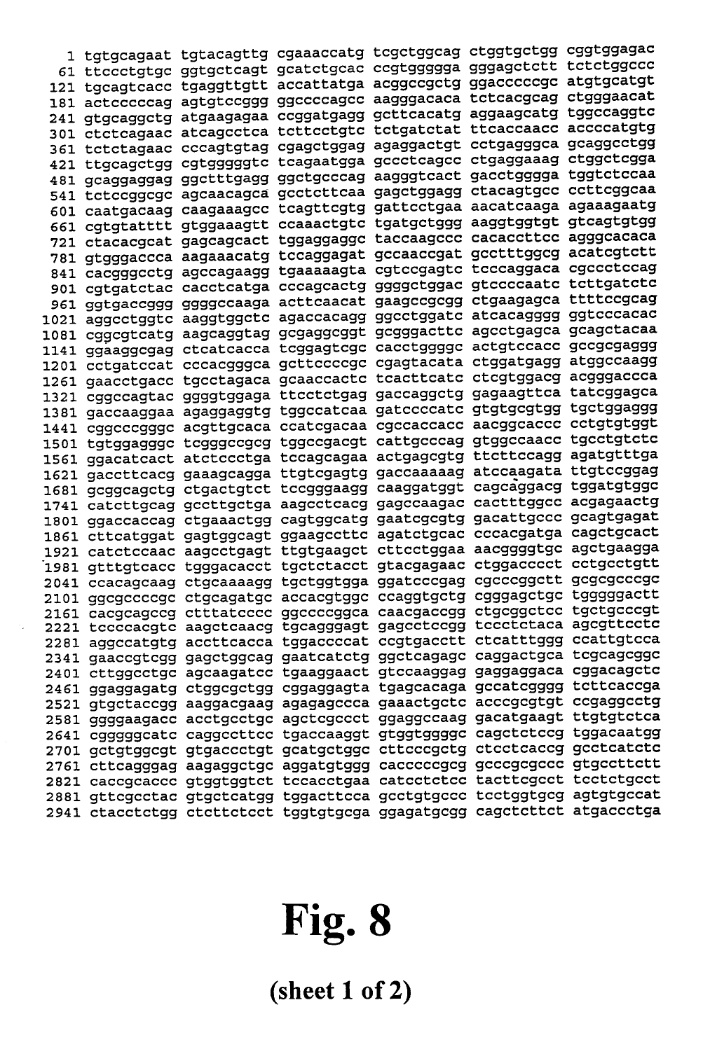

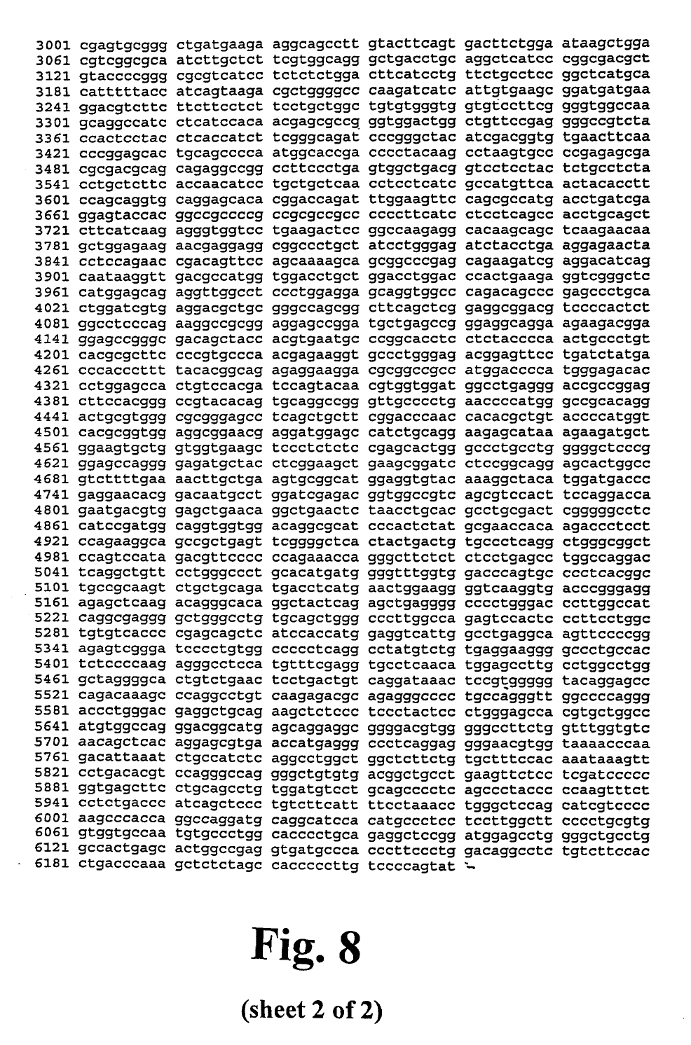

In yet another embodiment, LTRPC2 polypeptides from humans or from other organisms may be identified and isolated using oligonucleotide probes or degenerate polymerase chain reaction (PCR) primer sequences with an appropriate genomic or cDNA library. As will be appreciated by those in the art, the LTRPC2 unique NUDT9-H nucleic acid sequence comprising all or part of the carboxyl terminus of nucleotide sequences of SEQ ID NO:2 (FIG. 7) or SEQ ID NO:3 (FIG. 8), is particularly useful as a probe and/or PCR primer sequence. As is generally known in the art, preferred PCR primers are from about 15 to about 35 nucleotides in length, with from about 20 to about 30 being preferred, and may contain inosine as needed. The conditions for the PCR reaction are well known in the art.

In a preferred embodiment, LTRPC2 is a “recombinant protein” which is made using recombinant techniques, i.e. through the expression of a recombinant LTRPC2 nucleic acid. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or of amino acid substitutions, additions and deletions, as discussed below.

In a further embodiment, LTRPC2 variants may be recombinantly engineered by replacing one amino acid with another amino acid having similar structural and/or chemical properties, such as the replacement of a leucine with a serine, i.e., conservative amino acid replacements.

In a further embodiment substitutions, deletions, additions or any combination thereof may be used to make LTRPC2 variants. Generally these changes are done on a few amino acids to minimize the alteration of the molecule. However, larger changes may be tolerated in certain circumstances. When small alterations in the characteristics of the LTRPC2 polypeptide are desired, substitutions are generally made in accordance with the following Table 1:

TABLE 1

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In a further embodiment, substantial changes in function or in immunological identity are made by selecting substitutions that are less conservative than those shown in Chart 1. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine. The LTRPC2 variants of this embodiment exhibit one or more properties of the LTRPC2 polypeptides originally defined herein.

In a further emodiment, the variants typically exhibit the same qualitative biological activity and will elicit the same immune response as the naturally-occurring analogue, although variants also are selected to modify the characteristics of the LTRPC2 polypeptides as needed. Alternatively, the variant may be designed such that the biological activity of the LTRPC2 polypeptides is altered. For example, glycosylation sites may be altered or removed. The proteins enocoded by the nucleic acid variants exhibit at least one of the novel LTRPC2 polypeptide properties defined herein.

The proteins enocoded by nucleic acid variants exhibit at least one of the novel LTRPC2 polypeptide properties defined herein.

As used herein, “LTRPC2 nucleic acids” or their grammatical equivalents, refer to nucleic acids, that encode LTRPC2 polypeptides exhibiting one or more of the novel LTRPC2 polypeptide properties previously described. The LTRPC2 nucleic acids exhibit sequence homology to SEQ ID NO:2 (FIG. 7) or SEQ ID NO:3 (FIG. 8) where homology is determined by comparing sequences or by hybridization assays.

An LTRPC2 nucleic acid encoding an LTRPC2 polypeptide is homologous to the cDNA forth in FIG. 7 (SEQ ID NO:2) and/or the genomic DNA set forth in FIG. 8 (SEQ ID NO:3). Such LTRPC2 nucleic acids are preferably greater than about 75% homologous, more preferably greater than about 80%, more preferably greater than about 85% and most preferably greater than 90% homologous. In some embodiments the homology will be as high as about 93 to 95 or 98%. Homology in this context means sequence similarity or identity, with identity being preferred. A preferred comparison for homology purposes is to compare the sequence containing sequencing differences to the known LTRPC2 sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al, Nucl. Acid Res. 12:387-395 (1984), preferably using the default settings, or by inspection.

In a preferred embodiment, the % identity values used herein are generated using a PILEUP algorithm. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. EvoL 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

In preferred embodiment, a BLAST algorithm is used. BLAST is described in Altschul et al., J. Mol. BioL. 215:403-410, (1990) and Karlin et al., PNAS USA 90:5873-5787 (1993). A particularly useful BLAST program is the WU-BLAST-2, obtained from Altschul et al., Methods in Enzymology, 266:460-480 (1996); http://blast.wustl/edu/blast/README.html. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a preferred embodiment, “percent (%) nucleic acid sequence identity” is defined as the percentage of nucleotide residues in a candidate sequence that are identical with the nucleotide residue sequences of SEQ ID NO:2 (FIG. 7) and/or of SEQ ID NO:3 (FIG. 8). A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleosides than those of SEQ ID NO:2 (FIG. 7) and/or SEQ ID NO:3 (FIG. 8), it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

As described above, the LTRPC2 nucleic acids can also be defined by homology as determined through hybridization studies. Hybridization is measured under low stringency conditions, more preferably under moderate stringency conditions, and most preferably, under high stringency conditions. The proteins encoded by such homologous nucleic acids exhibit at least one of the novel LTRPC2 polypeptide properties defined herein. Thus, for example, nucleic acids which hybridize under high stringency to a nucleic acid having the sequence set forth as SEQ ID NO:2 (FIG. 7) or SEQ ID NO:3 (FIG. 8) and their complements, are considered LTRPC2 nucleic acid sequences providing they encode a protein having an LTRPC2 property.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional examples of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1 % bovine serum albumin/0.1% Ficoll/0.1% polyvinylpyrrolidone/50 mM sodium phosphate buffer at pH 6.5 with 750 mM sodium chloride, 75 mM sodium citrate at 42° C; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC. (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC. (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC. at about 37-50° C. The skilled artisan will recognize how to adjust the temperature, ionic strength, etc. as necessary to accommodate factors such as probe length and the like. Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

In another embodiment, less stringent hybridization conditions are used; for example, moderate or low stringency conditions may be used, as are known in the art. For additional details regarding stringency of hybridization reactions, see Ausubel et al., Current Protocols in Molecular Biology, Wiley Interscience Publishers, (1995).

The LTRPC2 nucleic acids, as defined herein, may be single stranded or double stranded, as specified, or contain portions of both double stranded or single stranded sequence. As will be appreciated by those in the art, the depiction of a single strand (“Watson”) also defines the sequence of the other strand (“Crick”); thus the sequences described herein also include the complement of the sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

The LTRPC2 nucleic acids, as defined herein, are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Homologs and alleles of the LTRPC2 nucleic acid molecules are included in the definition. Genetically modified LTRPC2 nucleic acid molecules are further included in this definition.

The full-length native sequence LTRPC2 gene (SEQ ID NO:3), or portions thereof, may be used as hybridization probes for a cDNA library to isolate the full-length LTRPC2 gene from other multicellular eukaryotic species, or to isolate still other genes (for instance, those encoding naturally-occurring variants of the LTRPC2 polypeptide or the LTRPC2 polypeptide from other multicellular eukaryotic species) which have a desired sequence identity to a particular LTRPC2 nucleotide coding sequence. Optionally, the length of the probes will be about 20 through about 50 bases. The hybridization probes may be derived from the nucleotide sequences of SEQ ID NO:2, the nucleotide sequences of SEQ ID NO:3, or from genomic sequences including promoters, enhancer elements and introns of particular native nucleotide sequences of LTRPC2. By way of example, a screening method will comprise isolating the coding region of an LTRPC2 gene using the known DNA sequence to synthesize a selected probe of about 40 bases.

Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the LTRPC2 gene of the invention can be used to screen libraries of human cDNA, genomic DNA or mRNA to determine which members of such libraries the probe hybridizes to. Hybridization have been previously described below.

The probes may also be employed in PCR techniques to generate a pool of sequences for identification of closely related LTRPC2 nucleotide coding sequences. Nucleotide sequences encoding LTRPC2 polypeptides can also be used to construct hybridization probes for mapping the gene which encodes that LTRPC2 and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries

In another embodiment, DNA encoding the LTRPC2 polypeptide may be obtained from a cDNA library prepared from tissue believed to possess the LTRPC2 mRNA and to express it at a detectable level. Accordingly, human LTRPC2 DNA can be conveniently obtained from a cDNA library prepared from human tissue, or a cDNA spleen library prepared from human spleen tissue. The LTRPC2-encoding gene may also be obtained from a multicellular eukaryotic genomic library or by oligonucleotide synthesis.

Libraries can be screened with probes (such as antibodies to LTRPC2 DNA or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding LTRPC2 is to use PCR methodology [Sambrook et al., supra; Dieffenbach et al., PCR Primer: A Laboratory Manual (Cold Spring Harbor Laboratory Press, 1995)].

The examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ADPR, biotinylation or enzyme labeling. Hybridization conditions, including moderate stringency and high stringency, are provided in Sambrook et al., supra, and have been described previously.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs such as ALIGN, DNAstar, BLAST, BLAST2 and INHERIT which employ various algorithms to measure homology, as has been previously described.

Nucleic acid encoding LTRPC2 polypeptides, as defined herein, may be obtained by screening selected cDNA or genomic libraries using all or part of the nucleotide sequences of SEQ ID NO:2 (FIG. 7) or of SEQ ID NO:3 (FIG. 8). Conventional primer extension procedures as described in Sambrook et al., supra, are used to detect precursors and processing intermediates of MRNA that may not have been reverse-transcribed into cDNA.

Nucleotide sequences (or their complement) encoding the LTRPC2 polypeptides have various applications in the art of molecular biology, including uses as hybridization probes, in chromosome and gene mapping, and in the generation of anti-sense RNA and DNA.

In another embodiment, the LTRPC2 nucleic acids, as defined herein, are useful in a variety of applications, including diagnostic applications, which will detect naturally occurring LTRPC2 nucleic acids, as well as screening applications; for example, biochips comprising nucleic acid probes to the LTRPC2 nucleic acids sequences can be generated. In the broadest sense, then, by “nucleic acid” or “oligonucleotide” or grammatical equivalents herein means at least two nucleotides covalently linked together.

In another embodiment, the LTRPC2 nucleic acid sequence of SEQ ID NO:2 (FIG. 7), as described above, is a fragment of a larger gene, i.e. it is a nucleic acid segment. “Genes” in this context include coding regions, non-coding regions, and mixtures of coding and non-coding regions. Accordingly, as will be appreciated by those in the art, using the sequences provided herein, additional sequences of LTRPC2 genes can be obtained, using techniques well known in the art for cloning either longer sequences or the full length sequences; see Maniatis et al., and Ausubel, et al., supra, hereby expressly incorporated by reference.

Once the LTRPC2 nucleic acid, as described above, is identified, it can be cloned and, if necessary, its constituent parts recombined to form the entire LTRPC2 gene. Once isolated from its natural source, e.g., contained within a plasmid or other vector or excised therefrom as a linear nucleic acid segment, the recombinant LTRPC2 nucleic acid can be further-used as a probe to identify and isolate other LTRPC2 nucleic acids, from other multicellular eukaryotic organisms, for example additional coding regions. It can also be used as a “precursor” nucleic acid to make modified or variant LTRPC2 nucleic acids.

In another embodiment, the LTRPC2 nucleic acid (e.g., cDNA or genomic DNA), as described above, encoding the LTRPC2 polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

A host cell comprising such a vector is also provided. By way of example, the host cells may be mammalian host cell lines which include Chinese hamster ovary (CHO), COS cells, cells isolated from human bone marrow, human spleen cells, cells isolated from human cardiac tissue, human pancreatic cells, and human leukocyte and monocyte cells. More specific examples of host cells include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad Sci. USA, 77:4216 (1980)); human pancreatic β-cells; mouse sertoli cells (TM4, Mather, Biol. Reprod., 23:243-251 (1980)); human lung cells (W138, ATCC. CCL 75); human liver cells (Hep G2, HB 8065); and mouse mammary tumor cells (MMT 060562, ATCC. CCL51). The selection of the appropriate host cell is deemed to be within the skill in the art. In the preferred embodiment, HEK-293 cells are used as host cells. A process for producing LTRPC2 polypeptides is further provided and comprises culturing host cells under conditions suitable for expression of the LTRPC2 polypeptide and recovering the LTRPC2 polypeptide from the cell culture.

In another embodiment, expression and cloning vectors are used which usually contain a promoter, either constitutive or inducible, that is operably linked to the LTRPC2-encoding nucleic acid sequence to direct MRNA synthesis. Promoters recognized by a variety of potential host cells are well known. The transcription of an LTRPC2 DNA encoding vector in mammalian host cells is preferably controlled by an inducible promoter, for example, by promoters obtained from heterologous mammalian promoters, e.g., the actin promoter or an immunoglobulin promoter, and from heat-shock promoters. Examples of inducible promoters which can be practiced in the invention include the hsp 70 promoter, used in either single or binary systems and induced by heat shock; the metallothionein promoter, induced by either copper or cadmium (Bonneton et al., FEBS Lett. 1996 380(1-2): 33-38); the Drosophila opsin promoter, induced by Drosophila retinoids (Picking, et al., Experimental Eye Research. 1997 65(5): 717-27); and the tetracycline-inducible full CMV promoter. Of all the promoters identified, the tetracycline-inducible full CMV promoter is the most preferred. Examples of constitutive promoters include the GAL4 enhancer trap lines in which expression is controlled by specific promoters and enhancers or by local position effects (http://www.fruitfly.org; http://www.astorg.u-strasbg.fr:7081); and the transactivator-responsive promoter, derived from E. coli, which may be either constitutive or induced, depending on the type of promoter it is operably linked to.

Transcription of a DNA encoding the LTRPC2 by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the LTRPC2 coding sequence, but is preferably located at a site 5′ from the promoter.

The methods of the invention utilize LTRPC2 polypeptides or nucleic acids which encode LTRPC2 polypeptides for identifying candidate bioactive agents which bind to LTRPC2, which modulate the activity of LTRPC2 ion channels, or which alter the expression of LTRPC2 within cells

The term “candidate bioactive agent” as used herein describes any molecule which binds to LTRPC2, modulates the activity of an LTRPC2 ion channel, and/or alters the expression of LTRPC2 within cells. A molecule, as described herein, can be an oligopeptide, small organic molecule, polysaccharide, or polynucleotide, etc. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 100 and less than about 2,500 daltons (D). Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of multicellular eucaryotic proteins may be made for screening in the methods of the invention. Particularly preferred in this embodiment are libraries of multicellular eukaryotic proteins, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

In a preferred embodiment, anti-sense RNAs and DNAs can be used as therapeutic agents for blocking the expression of certain LTRPC2 genes in vivo. It has already been shown that short antisense oligonucleotides can be imported into cells where they act as inhibitors, despite their low intracellular concentrations caused by their restricted uptake by the cell membrane. (Zamecnik et al., (1986), Proc. Natl. Acad Sci. USA 83:4143-4146). The anti-sense oligonucleotides can be modified to enhance their uptake, e.g. by substituting their negatively charged phosphodiester groups by uncharged groups. In a preferred embodiment, LTRPC2 anti-sense RNAs and DNAs can be used to prevent LTRPC2 gene transcription into mRNAs, to inhibit translation of LTRPC2 mRNAs into proteins, and to block activities of preexisting LTRPC2 proteins.

As used herein, a multivalent cation indicator is a molecule that is readily permeable to a cell membrane or otherwise amenable to transport into a cell e.g., via liposomes, etc., and upon entering a cell, exhibits a fluorescence that is either enhanced or quenched upon contact with a multivalent cation. Examples of multivalent cation indicators useful in the invention are set out in Haugland, R.P. Handbook of Fluorescent Probes and Research Chemicals., 6th ed. Molcular Probes, Inc Eugene, Oreg., pp. 504-550 (1996); (http://www.probes.com/handbook/sections/2000.html), incorporated herein by reference in its entirety.

In a preferred embodiment for binding assays, either LTRPC2 or the candidate bioactive agent is labeled with, for example, a fluorescent, a chemiluminescent, a chemical, or a radioactive signal, to provide a means of detecting the binding of the candidate agent to LTRPC2. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound LTRPC2. As known in the art, unbound labeled streptavidin is removed prior to analysis. Alternatively, LTRPC2 can be immobilized or covalently attached to a surface and contacted with a labeled candidate bioactive agent. Alternatively, a library of candidate bioactive agents can be immobilized or covalently attached to a biochip and contacted with a labeled LTRPC2. Procedures which employ biochips are well known in the art.

In a preferred embodiment, the ion permeabilty of LTRPC2 is measured in intact cells, preferably HEK-293 cells, which are transformed with a vector comprising nucleic acid encoding LTRPC2 and an inducible promoter operably linked thereto. Endogenous levels of intracellular ions are measured prior to inducement and then compared to the levels of intracellular ions measured subsequent to inducement. Fluorescent molecules such as fura-2 can be used to detect intracellular ion levels. LTRPC2 permeability to Ca2+ and to other multivalent cations can be measured in this assay.

In a preferred embodiment for screening for candidate bioactive agents which modulate expression levels of LTRPC2 within cells, candidate agents can be used which wholly suppress the expression of LTRPC2 within cells, thereby altering the cellular phenotype. In a further preferred embodiment, candidate agents can be used which enhance the expression of LTRPC2 within cells, thereby altering the cellular phenotype. Examples of these candidate agents include antisense cDNAs and DNAs, regulatory binding proteins and/or nucleic acids, as well as any of the other candidate bioactive agents herein described which modulate transcription or translation of nucleic acids encoding LTRPC2.

In one embodiment, the invention provides antibodies which specifically bind to unique epitopes on the LTRPC2 polypeptide, e.g., unique epitopes of the protein comprising amino acids from 1 through about 1503 of SEQ ID NO:1 (FIG. 6).

In another embodiment, the invention provides an antibody which specifically binds to epitopes from three extracellular domains comprising sequences 774-793 or 892-899 or 957-1023 (FIG. 6).

The anti-LTRPC2 polypeptide antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the LTRPC2 polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The anti-LTRPC2 polypeptide antibodies may further comprise monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the LTRPC2 polypeptide or a fusion protein thereof Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against an LTRPC. polypeptide. Preferably, the binding specificity of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The anti-LTRPC2 polypeptide antibodies may further comprise monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. In vitro methods are also suitable for preparing monovalent antibodies. Digestion of antibodies to produce fragments thereof, particularly, Fab fragments, can be accomplished using routine techniques known in the art.

The anti-LTRPC2 polypeptide antibodies may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. MoL Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by the introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The anti-LTRPC2 polypeptide antibodies may further comprise heteroconjugate antibodies. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a further embodiment, the anti-LTRPC2 polypeptide antibodies may have various utilities. For example, anti-LTRPC2 polypeptide antibodies may be used in diagnostic assays for LTRPC2 polypeptides, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Further, LTRPC2 antibodies may be used in the methods of the invention to screen for their ability to modulate the permeability of LTRPC2 channels to multivalent cations.

### EXAMPLES

Commercially available reagents referred to in the examples were used according to manufacturer's instructions unless otherwise indicated.

### Example 1

RT-PCR and Northern Blot Analysis of Expression.

For PCR analysis of LTRPC2 expression, the oligos used were CAGTGTGGCTACACGCATGA and TCAGGCCCGTGAAGACGATG to produce a 138 bp band. For analysis of NUDT9 expression, the oligos used were GGCAAGACTATAAGCCTGTG and ATAATGGGATCTGCAGCGTG to produce a 252 base pair band. Amplification conditions used were 95 degree melting, 55 degree annealing, and 72 degree extension for 25 cycles. All libraries screened were from Life Technologies. For northern blots, single stranded probes were constructed with the NotI/BglII fragment of the human LTRPC2 sequence as template using an Ambion StripEZ T7 RNA probe kit according to the manufacturers instructions. RNA was extracted from the indicated cell lines using the FastTrack mRNA extraction kit (Invitrogen), and transferred to nylon membranes using standard methods. Hybridizations were performed using Ultrahyb hybridization buffer (Ambion) at 65-68 degrees and otherwise standard methods.

### Example 2

Cloning and Sequence Analysis of LTRPC2 and NUDT9.

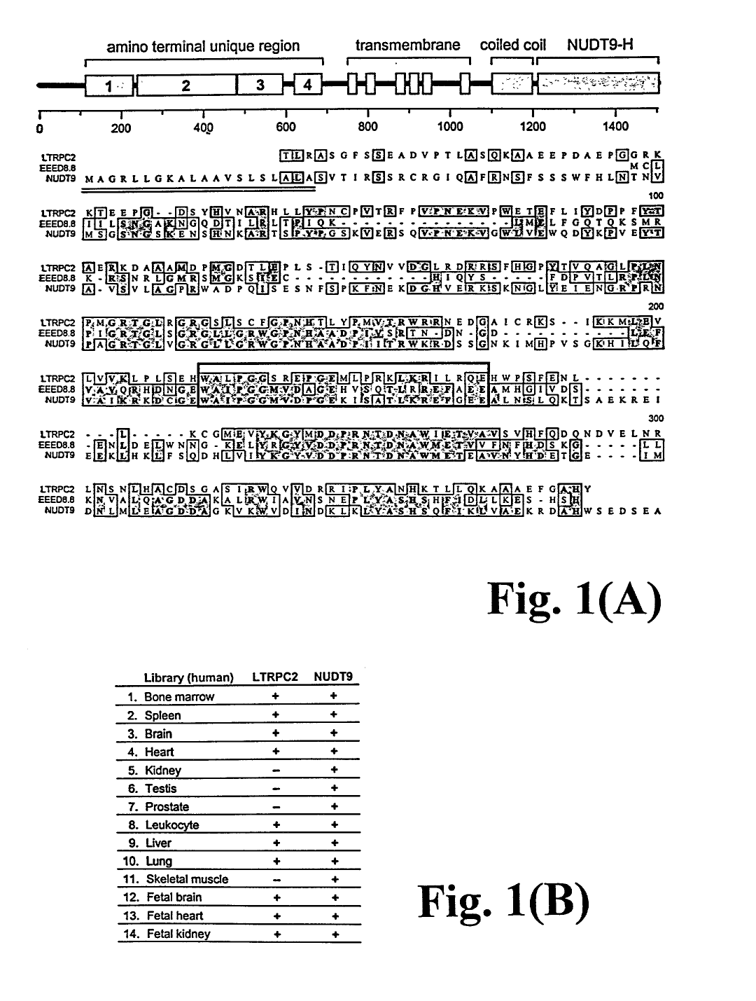

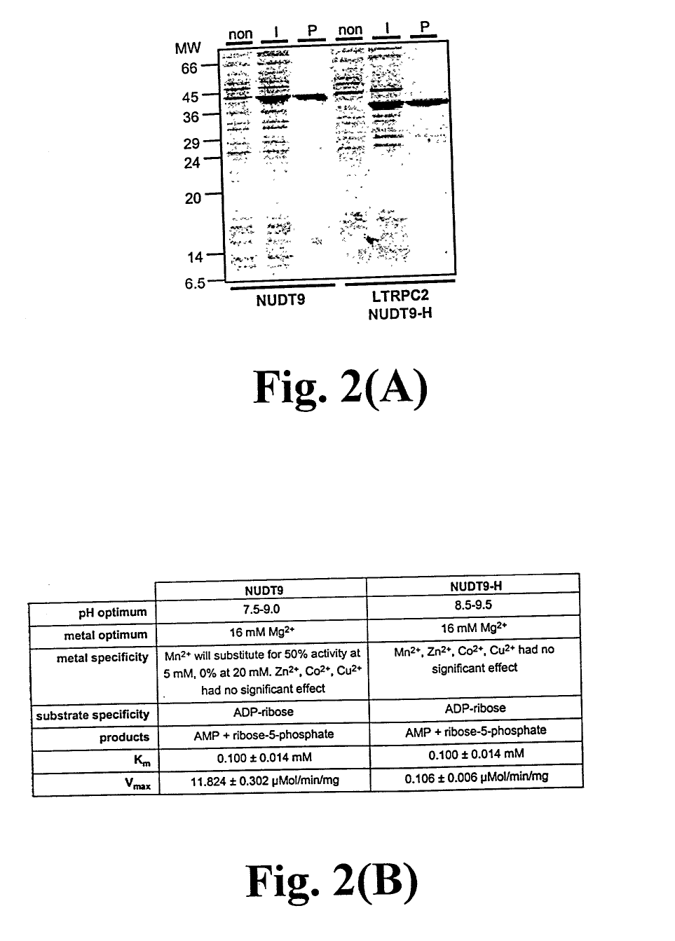

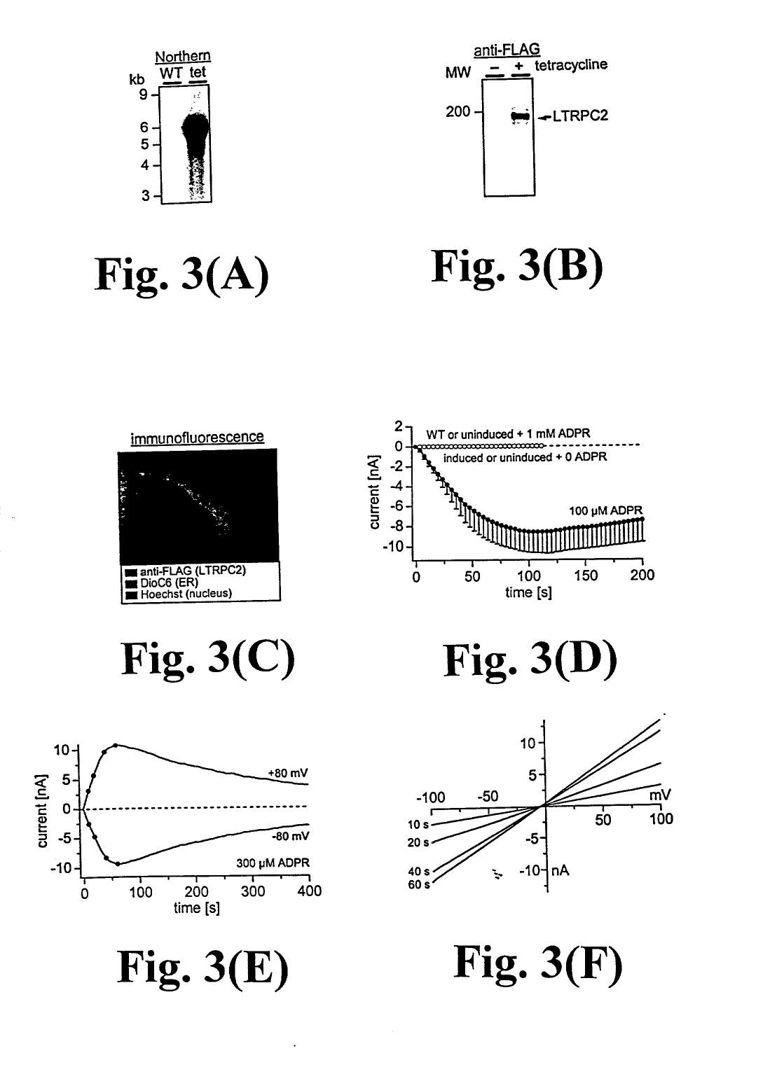

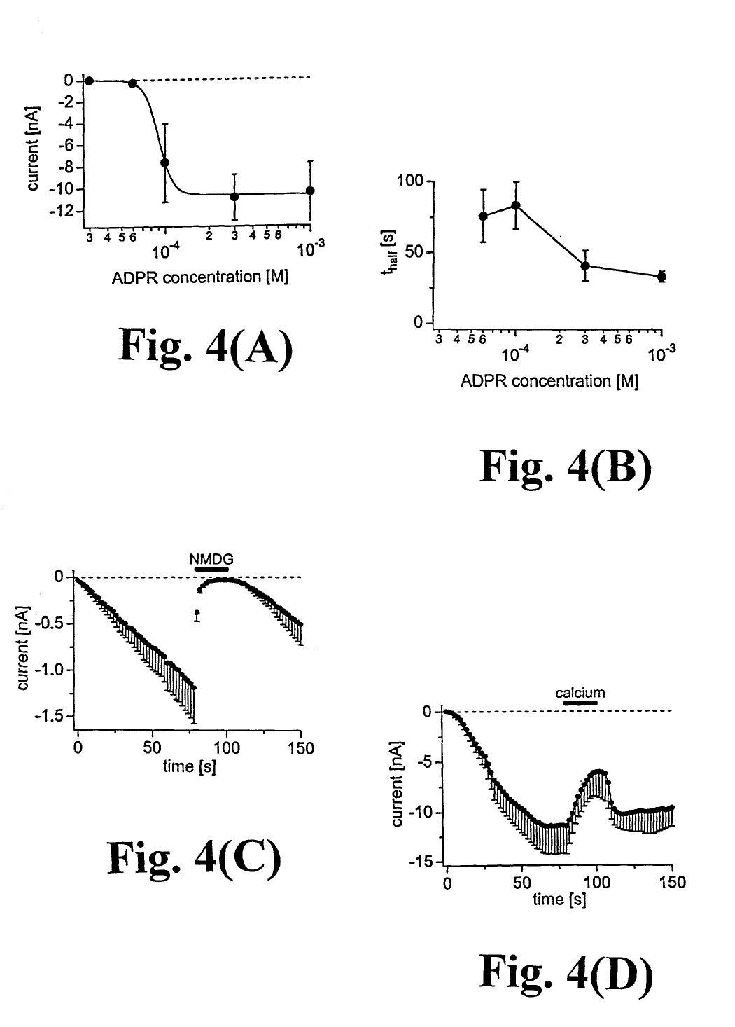

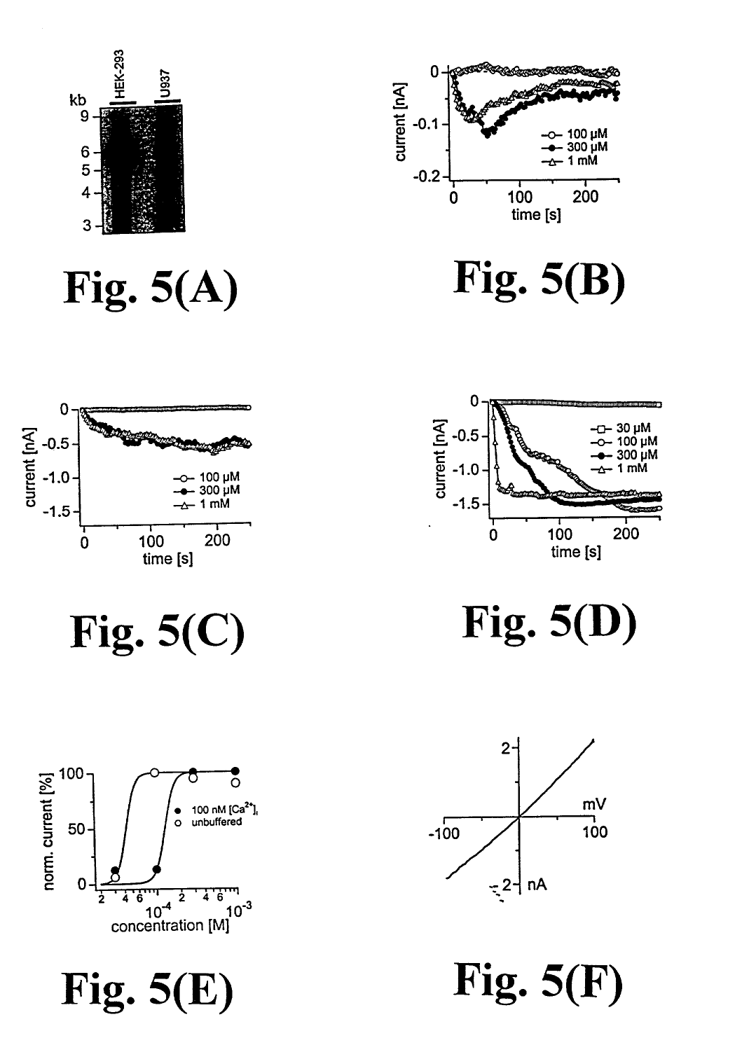

The genetrapper II solution hybridization method (Life Technologies) was used to isolate both LTRPC2 and NUDT9 cDNA's. For LTRPC2, five PCR positive colonies were obtained from the leukocyte library that was positive for LTRPC2 expression by RT-PCR in FIG. 1b, and the longest of these (4.0 kb) was sequenced. For NUDT9, 35 colonies were obtained from the spleen library that was positive for NUDT9 expression in FIG. 1b. Eight of these were end-sequenced to confirm that they represented the same transcript and one was fully sequenced in both directions.

### Example 3

Construction of a FLAG-Tagged LTRPC2 Expression Construct.

Brain cDNA was purchased from Clontech and used to obtain by RT-PCR the LTRPC2 coding sequence not present in the 4.0 kb fragment isolated by cDNA cloning. This sequence extended from the internal NotI site present in LTRPC2 to the stop codon, and included an additional Kpnl site just internal to the stop codon, thereby adding an additional two amino acids (glycine and threonine) to the 3′ end of LTRPC2, followed by a stop codon and a Spel site just beyond the stop codon. This RT-PCR fragment was ligated onto the 4.0 Kb cDNA using the Noti site and Spel sites, producing a full length LTRPC2 coding sequence. The internal NotI site in this full-length LTRPC2 template was then removed by site-directed mutagenesis, and PCR was used to generate a LTRPC2 expression construct containing a NotI site at the 5′ end internal to the initiating methionine. This construct was subcloned into a modified pCDNA4/TO vector containing a Kozak sequence, initiating methionine, FLAG tag, and polylinker including a NotI site in appropriate frame with the FLAG tag and a 3′ SpeI site. This produced an expression plasmid that yielded a protein with the following predicted sequence:

MGDYKDDDDKRPLA—followed by the LTRPC2 coding sequence beginning at amino acid 3 and extending to amino acid 1503—followed by GT and then the stop codon. Sequencing of the full-length LTRPC2 construct showed four single base pair differences with the original LTRPC2/TrpC7 sequence. Three of these did not change the predicted amino acid sequence, while the fourth introduced a glycine for serine substitution at amino acid 1367 relative to the published LTRPC2/TrpC7 sequence. This was interpreted as a possible polymorphic form of LTRPC2/TprC7, therefore an otherwise identical “wild type” LTRPC2 expression construct was also produced. FLAG-LTRPC2 and FLAG-LTRPC2(S1367G) constructs were used in each of the various types of experiments presented, and were indistinguishable in terms of their biochemical and biophysical behavior.

### Example 4

Construction of E. Coli Expression Constructs for NUDT9 and NUDT9-H Region of LTRPC2.

A full-length coding sequence for NUDT9 was produced by PCR to place an NcoI site at the 5′ end and an Noti site at the 3′end, and subcloned into the pET-24d T7 expression vector from Novagen. For the LTRPC2 NUDT9 homology region, a construct was made by PCR to include an NcoI site, an artificial start codon, amino acids 1197-1503, a stop codon, and a 3′ NotI site. This was also subcloned into pET-24d. Both a wild type LTRPC2 NUDT9 homology region and an LTRPC2(S1367G) NUDT9 homology region construct were evaluated and were indistinguishable in terms of enzymatic activity in vitro.

### Example 5

E. Coli Expression and Purification of NUDT9 and the NUDT9 Homology Region of LTRPC2.

BL21 (DE3) cells containing the respective expression plasmids were grown at 37° C. in LB broth on a shaker to an A600 of about 0.6 and induced by the addition of isopropyl-b-D-thiogalactopyranoside to a concentration of 1 mM. The cells were grown for an additional 4 h, harvested, washed by suspension in isotonic saline, centrifuged in pre-weighed centrifuge tubes, and the packed cells were stored at −80 ° C. The expressed protein leaked out of the frozen and thawed cells when washing them in 50 mM Tris, pH 7.5, 1 mM EDTA, 0.1 mM dithiothreitol. Most endogenous proteins remained within the cells, resulting in an extract enriched for the expressed enzymes. In the case of NUDT9, enzyme was extracted in the freeze-thaw fraction and ammonium sulfate was added to 35% final concentration. The precipitate was discarded after centrifugation and ammonium sulfate was added to the supernatant to a final concentration of 50%. The precipitate was collected by centrifugation, dissolved, then chromatographed on a gel filtration column (Sephadex G-100). The active fractions containing the majority of the enzyme were pooled, concentrated by centrifugation in an Amicon Centriprep30, dialyzed, and chromatographed on DEAE-sepharose. The purified enzyme was concentrated from the pooled active fractions again using an Amicon Centriprep30. For the NUDT9 homology region of LTRPC2, the protein was extracted in the freeze-thaw fraction and ammonium sulfate was added to 35% final concentration and centrifuged. The precipitate was dissolved, dialyzed, and chromatographed on DEAE-sepharose. The purified enzyme was concentrated from the pooled active fractions by precipitation with 70% ammonium sulfate.

### Example 6

Assays for Nudix Type Activity of NUDT9 and NUDT9-H Region of LTRPC2.

Enzyme Assay: Enzyme velocities were quantified by measuring the conversion of a phosphatase-insensitive substrate, ADPR, to the phosphatase-sensitive products, AMP and ribose-5-phosphate. The liberated inorganic orthophosphate was measured by the procedure of Ames and DubinENRfu²⁷. The standard incubation mixture (50 ml) contained 50 mM Tris-Cl, pH 9.0, 16 mM MgCl₂, 2 mM ADPR, 0.2-1 milliunitsof enzyme and 4 units of alkaline intestinal phosphatase. After 30 min at 37° C., the reaction was terminated by the addition of EDTA and inorganic orthophosphate was measured. A unit of enzyme hydrolyzes 1 mmol of substrate per min under these conditions. Note that 2 moles of phosphate are liberated per mole of ADPR hydrolyzed. Product determination: The standard assay mixture (minus alkaline intestinal phosphatase) was incubated for 30 min at 37° C. and terminated by the addition of 50 ml of a mixture of four parts of Norit (20% packed volume) and one part of 7% HClO4 to remove adenine-containing nucleotides. After centrifugation, 50 ml was adjusted to an alkaline pH and incubated for an additional 30 min at 37° C. with alkaline intestinal phosphatase to hydrolyze the ribose-5-phosphate formed. The subsequent free phosphate was measured and compared to a control reaction that did not undergo Norit treatment. The stoichiometric relation between the two suggests the products are AMP and ribose-5-phosphate.

### Example 7

Construction of HEK-293 Cells Expressing Tetracycline-Regulated LTRPC2.

FLAG-LTRPC2 and FLAG-LTRPC2(S1367G) constructs in pCDNA4/TO were electroporated into HBEK-293 cells previously transfected with the pCDNA6/TR construct so as to express the tetracycline repressor protein. Cells were placed under zeocin selection, and zeocin-resistant clones were screened for inducible expression of a FLAG-tagged protein of the correct molecular weight. The clones with the lowest level of basal expression and the best overall level of protein expression after tetracycline or doxycycline treatment were chosen for further analysis.

### Example 8

SDS/PAGE, Immunoprecipitation, Immunoblotting and Immunofluorescence.

SDS/PAGE, immunoprecipitation, and immunoblotting were all performed using standard methods or as described in the figure legends. For immunofluorescence, after 24 h tetracycline induction, HEK-293 cells were fixed (4% paraformaldehyde, 20 min) and permeabilized (0.2 % triton X-100, 4 min) before sequential exposure to Hoechst (1 mg/ml, 2 min) and DioC6 (0.3 mg/ml, 2 min) (Molecular Probes). For anti-FLAG immunofluorescence, cells were then blocked (0.2 % fish-skin gelatin, 20 min) and probed with anti-FLAG (IBI-Kodak), followed by Alexa 568 goat anti-mouse IgG (Molecular Probes), both in 0.05% fish-skin gelatin, 30 min exposure time. Mounted samples were imaged using single emission filters (Texas Red, FITC., Hoechst).

### Example 9

Cell Culture.

Wild type and tetracycline-inducible HEK-293 FLAG-LTRPC2 expressing cells were cultured at 37° C./5% CO₂ in DMEM supplemented with 10% FBS and 2 mM glutamine. The medium was supplemented with blasticidin (5 μg/ml; Invitrogen) and zeocin (0.4 mg/ml; Invitrogen). Cells were resuspended in media containing 1 μg/ml tetracycline (Invitrogen) 24 hours before experiments.

### Example 10

Electrophysiology.

For patch-clamp experiments, cells grown on coverslips were transferred to the recording chamber and kept in a standard modified Ringer's solution of the following composition (in mM): NaCl 145, KCl 2.8, CaCl₂ 1, MgCl₂ 2, glucose 10, Hepes-NaOH 10, pH 7.2. Intracellular pipette-filling solutions contained (in mM): Cs-glutamate 145, NaCl 8, MgCl₂ 1, Cs-BAPTA 10, pH 7.2 adjusted with CsOH. In some experiments, BAPTA was omitted from the pipette solution and 100 μM fura-2 was added for the purpose of fluorimetric monitoring of intracellular Ca²⁺ concentration. Adenosine 5-diphospho (ADP)-ribose, cyclic ADPR, ADP, guanosine 5-diphospho (GDP)-glucose, GDP-mannose, uridine diphospho (UDP)-glucose, UDP-mannose, ADP-glucose, ADP-mannose, cytosine diphospho (CDP)-glucose, ribose-5-phosphate, adenosine 5-monophosphate (AMP), nicotinamide adenine dinucleotide (AND) and inositol 1,4,5-trisphosphate (InsP₃) were purchased from Sigma. The agonists were dissolved in the standard intracellular solution. Patch-clamp experiments were performed in the tight-seal whole-cell configuration at 21-25° C. High-resolution current recordings were acquired by a computer-based patch-clamp amplifier system (EPC-9, HEKA, Lambrecht, Germany). Patch pipettes had resistances between 2-4 MW after filling with the standard intracellular solution. Immediately following establishment of the 5 whole-cell configuration, voltage ramps of 50 ms duration spanning the voltage range of −100 to +100 mV were delivered from a holding potential of 0 mV at a rate of 0.5 Hz over a period of 200 to 400 seconds. All voltages were corrected for a liquid junction potential of 10 mV between external and internal solutions. Currents were filtered at 2.9 kHz and digitized at 100 μs intervals. Capacitive currents and series resistance were determined and corrected before each voltage ramp using the automatic capacitance compensation of the EPC-9. For analysis, the very first ramps prior to current activation were digitally filtered at 2 kHz, pooled and used for leak-subtraction of all subsequent current records. The low-resolution temporal development of currents at a given potential was extracted from the leak-corrected individual ramp current records by measuring the current amplitudes at voltages of −80 mV or +80 mV.

What is claimed is:

1. A method for screening for a candidate bioactive agent capable of binding to LTRPC2, said method comprising:
a) contacting an LTRPC2 protein or fragment thereof with said candidate agent; and
b) determining the binding of said candidate agent to said LTRPC2 protein or fragment thereof.

2. The method of claim 1 wherein a library of two or more of said candidate agents are contacted with said LTRPC2 protein or fragment thereof.

3. The method of claim 1 wherein said LTRPC2 protein comprises amino acids from 1 through about 1503 of SEQ ID NO:1.

4. The method of claim 1 wherein said LTRPC2 protein is encoded by a nucleic acid comprising sequences from 1 through about 4512 of SEQ ID NO:2.

5. A method for screening a candidate bioactive agent comprising
a) contacting an LTRPC2 channel with the candidate agent, and
b) detecting whether said agent modulates the multivalent cationic permeability of said LTRPC2 channel.

6. The method of claim 5 wherein said modulating activity opens said LTRPC2 channel.

7. The method of claim 5 wherein said modulating activity closes said LTRPC2 channel.

8. A method for screening for a candidate bioactive agent capable of modulating multivalent cation permeability of an LTRPC2 channel, said method comprising:
a) providing a recombinant cell comprising a recombinant nucleic acid comprising nucleic acid encoding LTRPC2 and an inducible promoter operably linked thereto which is capable of expressing said LTRPC2, and further comprising a multivalent cation indicator;
b) inducing said recombinant cell to express said LTRPC2;
c) contacting said recombinant cell with a multivalent cation and said candidate agent; and
d) detecting the intracellular levels of said multivalent cation with said indicator.

9. The method of claim 8 wherein said contacting is of said candidate agent followed by said multivalent cation.

10. The method of claim 8 wherein the modulating activity increases said multivalent cation permeability of said LTRPC2 channel;

11. The method of claim 8 wherein the modulating activity decreases said multivalent cation permeability of said LTRPC2 channel.

12. The method of claim 8 wherein said indicator comprises a fluorescent molecule.

13. The method of claim 12 wherein said fluorescent molecule comprises fura-2.

14. A method for measuring multivalent cation permeability of an LTRPC2 channel, said method comprising:
a) providing a recombinant cell wherein said cell comprises a recombinant nucleic acid which expresses LTRPC2 and further comprises a multivalent cation indicator;
b) contacting said recombinant cell with a multivalent cation which selectively interacts with said indicator to generate a signal; and
c) measuring the intracellular levels of said multivalent cation by detecting said indicator signal.

15. The method of claim 14 wherein said indicator comprises a fluorescent molecule.

16. The method of claim 15 wherein said fluorescent molecule comprises fura-2.

17. The method of claim 14 further comprising contacting said recombinant cell with a candidate bioactive agent.

18. The method of claim 17 wherein said modulating activity increases said multivalent cation permeability of said LTRPC2 channel;

19. The method of claim 17 wherein said modulating activity decreases said multivalent cation permeability of said LTRPC2 channel.

20. The method of claim 17 wherein said measuring further comprises comparing said intracellular multivalent cation levels to intracellular multivalent cation levels in a cell which does not express recombinant LTRPC2.

21. The method of claim 17 wherein said measuring further comprises comparing said intracellular multivalent cation levels to intracellular multivalent cation levels in a cell which does not express recombinant LTRPC2 but which is in contact with said candidate agent.

22. A method for screening for a candidate bioactive agent capable of modulating expression of an LTRPC2 protein or fragment thereof comprising:
a) providing a recombinant cell capable of expressing a recombinant nucleic acid encoding an LTRPC2 protein;
b) contacting said cell with said candidate agent; and
c) determining the effect of said candidate agent on the expression of said recombinant nucleic acid.

23. The method of claim 22 wherein said determining is a phenotype of said cell.

24. The method of claim 22 wherein the determining comprises determining the level of expression of LTRPC2 in the presence of said candidate agent and comparing said level of expression to endogenous LTRPC2 levels.

25. The method of claims 1, 5, 8, 14, and 22, wherein said candidate agent comprises a small molecule, protein, polypeptide or nucleic acid.
